(19) United States
(12) Patent Application Publication
(10) Pub. No.: US 2023/0290436 A1
(43) Pub. Date: Sep. 14, 2023

(54) TRAINING METHOD AND MODEL FOR PREDICTING INHIBITORS OF DRUGS METABOLIZING ENZYMES

(71) Applicants: INSERM (INSTITUT NATIONAL DE LA SANTÉ ET DE LA RECHERCHE MÉDICALE), Paris (FR); UNIVERSITE DE NANTES, Nantes (FR); ASSISTANCE PUBLIQUE HOPITAUX DE PARIS, Paris (FR); UNIVERSITÉ DE PARIS, Paris (FR); CENTRE NATIONAL DE LA RECHERCHE SCIENTIFIQUE - CNRS -, Paris (FR); UNIVERSITÉ DE LORRAINE, Nancy (FR); SORBONNE UNIVERSITE, Paris (FR)

(72) Inventors: Maria MITEV, Paris (FR); Marie-Anne LORIOT, Paris Cedex 06 (FR); Arnaud NICOT, Nantes Cedex 1 (FR); Elodie GOLDWASER, Evry-Courcouronnes (FR); Christian JELSCH, Vandoeuvre les Nancy Cedex (FR); Catherine LAURENT, Paris (FR)

(21) Appl. No.: 18/006,030

(22) PCT Filed: Jul. 23, 2021
(86) PCT No.: PCT/EP2021/070646

§ 371 Date: Jan. 19, 2023

(30) Foreign Application Priority Data
Jul. 24, 2020 (EP) 20305852.4

(51) Int. Cl. G16B 15/30; G16B 40/20; G06F 18/2411

(52) U.S. Cl. CPC G16B 15/30; G16B 40/20; G06F 18/2411

(57) ABSTRACT

The present invention relates to the prediction of drug metabolizing enzymes (DME) inhibitors. Inhibition of DMEs leads to adverse drug-drug interaction, hence predicting inhibition of DMEs by determined molecules is critical for preventing drug toxicity. Inventors elaborated a protocol of integrated in silico protein structure-based and machine learning approach to predict inhibition of DMEs. In particular, the present invention relates to a method for training a model for predicting inhibition of DMEs, comprising a selection of a number of descriptors among an initial set comprising physicochemical descriptors and binding energies on at least one enzyme configuration, and a training of a classification model on a learning database of known inhibitors or non-inhibitors based on the selected descriptors as inputs. This approach successfully predicted inhibition of CYP2C9, CYP2D6, SULT1A1, SULT1A3 and UGT1 A1.

### TECHNICAL FIELD

The present disclosure relates to the training and use of a classification model to predict the inhibiting character of a molecule on a determined Drug Metabolizing Enzymes (DME), in particular an enzyme pertaining to the family of cytochromes P450 (CYP), sulfotransferases (SULT) and UDP-glucuronosyltransferases.

### TECHNICAL BACKGROUND

Drug metabolizing enzymes play a key role in the metabolism of endogenous molecules, xenobiotics and drugs introduced into the human body. While their principal role is to detoxify organisms by modifying endogenous and exogenous compounds for a rapid excretion, such as pollutants or drugs, in some cases they render their substrates more toxic thereby inducing severe side effects and adverse drug reactions. Phase I DMEs catalyze oxidative reactions leading to metabolites that may be either excreted or additionally modified by the phase II DMEs catalyzing conjugation reactions. In some cases, phase II DMEs can directly modify a compound without passing through the phase I DMEs. Inhibition of DMEs is a complex process because it can correspond to a competitive inhibition in the active site, a modification of the substrate or metabolite flux between the active site and outside of the enzyme or an inhibition by a drug itself or by its metabolites (time-dependent inhibition) leading then to adverse drug-drug interactions. Thus, predicting potential DMEs inhibition is critical for preventing drug toxicity.

Among DMEs, Cytochrome P450 (CYP) is a superfamily of oxidizing enzymes responsible for the metabolism of drugs, xenobiotics and endogenous molecules. It is estimated that about 75% of marketed drugs are metabolized by CYPs with six major isoforms: 1A2, 2C8, 2C9, 2C19, 2D6 and 3A4. CYP inhibition leads to decreased drug elimination, which is the major cause of adverse drug-drug interactions. In some cases, CYP oxidation leads to toxic metabolites. There is therefore a need to identify potential inhibition of CYP enzymes for clinical drug treatment and early-stage drug discovery.

Among DMEs, another family of enzymes that metabolize a large number of drugs are sulfotransferases (SULTs). SULTs catalyze the sulfoconjugation from the co-factor 3′-Phosphoadenosine 5′-Phosphosulfate (PAPS) to a hydroxyl or amino group of the substrate by executing a nucleophilic attack. At high concentrations some substrates inhibit the enzyme and dead-end complexes with bound inactive cofactor PAP have been identified. Sulfoconjugation usually facilitates excretion, but in some particular cases the pharmacological activity of some drugs increases (e.g., the hypotensive prodrug minoxidil becomes fully active after sulfate conjugation). Further, SULTs can convert some chemicals to carcinogens or to activators of promutagens by creating highly reactive sulfate esters that can bind covalently to DNA (e.g. 7,12-dimethylbenz(a) anthracene). SULTs that are responsible for the metabolism of small endogenous compounds and xenobiotics are localized in the cytosol. Four families of human SULTs have been identified by now, SULT1, SULT2, SULT4 and SULT6. Among SULT1 family, SULT 1A1 metabolizing a wide variety of compounds like phenols, sex steroid hormones (estrogens), thyroid hormones and drugs (e.g. minoxidil, paracetamol, 17α-ethinylestradiol), is the most expressed one (found in liver, intestine, kidney, thyroid, platelets).

Last, UDP-glucuronosyltransferases (UGTs) belong to the uridine diphosphate glucuronosyltransferase gene family. UGTs catalyze the covalent addition of glucuronic acid sugar moieties to a host of therapeutics and environmental toxins, as well as to a variety of endogenous steroids and other signaling molecules. UGT-catalyzed glucuronidation is thought to account for up to 35% of the phase II drug metabolism reactions. Three main isoforms, UGT 2B7, UGT 1A4 and UGT 1A1, are responsible for drugs modification of 35%, 20%, and 15% of the drugs metabolized by UGTs, respectively.

In order to predict the inhibiting character of a molecule over a DME, approaches have been proposed based on classification models.

In particular, the publication by V. Y. Martiny et al. “Integrated structure- and ligand-based in silico approach to predict inhibition of cytochrome P450 2D6”, in Bioinformatics 31(24), 3930-3937, doi: 10.1093/bioinformatics/btv486, 26 Aug. 2015, discloses an in silico approach for the prediction of CYP2D6, in which three learning algorithms, support vector machine, Random Forest and Naive Bayesian, have been trained and tested to predict inhibition of cytochrome P450 2D6.

More specifically, this article discloses performing various molecular dynamics simulations (MD) of CYP2D6 on one apo structure PDB ID 2F9Q and one holo structure co-crystallized with prinomastat in order to identify the binding site conformations best predicting the binding energies.

Then, each classification model was trained on a learning database comprising 343 inhibitors and 3002 inhibitors of CYP2D6, using as input descriptors for a given molecule a set of descriptors comprising extended connectivity fingerprints (ECFPs), and protein-ligand binding energies calculated on the best MD receptor conformation.

Those inhibition models were able to predict CYP2D6 inhibition with an accuracy of 78% on the training set and 75% on the external validation set. This method however suffers some limitations. First, the important number of descriptors used in this model (up to 2000), and the type of descriptors (ECFPs), does not allow understanding why a molecule is predicted as inhibitor or non-inhibitor or the main factors that impact the inhibiting character of a molecule.

Furthermore, the important number of descriptors makes the training and the use of the classification model slow because of the computational time needed to compute each descriptor for a given molecule.

Last, the model uses as descriptor a single binding energy computed for a single conformation of the enzyme. It can be anticipated that using a higher number of binding energies corresponding to various conformations could improve the performances of the prediction model.

### SUMMARY OF THE INVENTION

In view of the above, the invention aims at proposing a model for predicting inhibition of at least one drug metabolizing enzyme, and in particular of the type CYP, SULT or UGT, having improved performance.

Another aim of the invention is to propose a model which training and use is accelerated.

Another aim of the invention is to propose a model that can help better understand the inhibiting factors of an enzyme by a molecule.

To this end, a method for training a model for predicting inhibitors of a determined CYP, SULT or UGT enzyme in disclosed, implemented by a training device comprising a computer and a memory storing a training dataset comprising a number of molecules known as being inhibitor or non-inhibitor of the determined enzyme, the method comprising:
selecting, from an initial set of molecular descriptors comprising physicochemical molecular descriptors and at least one binding energy on at least one conformation of the determined enzyme, a subset of descriptors, based on the relative importance of the descriptors in predicting the inhibiting character of a molecule,
performing a supervised training, over the training dataset, of a classification model configured to receive as input a vector formed of the subset of molecular descriptors computed on a molecule, and to output an indication of the inhibiting character of the molecule on the determined enzyme.

In embodiments, the determined enzyme is selected among the group consisting of:
CYP 2C9
CYP 2D6
SULT 1A1,
SULT 1A3, and
UGT 1A1.

In embodiments, selecting descriptors based on their relative importance comprises training a plurality of random forest models on the learning dataset, computing a Gini importance index of all descriptors of the set, and selecting the descriptors having highest Gini importance.

In embodiments, the determination of the number of descriptors to select based on their relative importance comprises computing the average balanced accuracy of a plurality of random forest models with multiple sets of descriptors having a varying number of descriptors, and selecting the number of descriptors maximizing the balanced accuracy.

In embodiments, the method comprises a step, prior to the selection step of removing, from the initial set of descriptors:
highly correlated descriptors,
descriptors having missing or infinite values on data of the training dataset, and
descriptors having a variance below a determined threshold over the training dataset.

In embodiments, the classification model is a random forest model or a Support Vector Machine model.

According to another object, a classification model configured for predicting whether a molecule is inhibitor of a predetermined enzyme id disclosed, wherein the classification model is obtained by training on a training dataset in accordance with the method according to the above description.

In embodiments, the classification model may be formed of:
a first classifier formed by a random forest model trained according to the above description,
a second classifier formed a Support Vector Machine model trained according to the above description, and
a third classifier indicating whether a molecule is inhibitor of the enzyme based on the comparison of the lowest binding energy computed for a plurality of conformations of the enzyme with at least one threshold,
   the output of the model being a majority vote over the three classifiers.

A method for predicting whether a candidate molecule is inhibitor of a predetermined enzyme is also disclosed, comprising:
computing a set of molecular descriptors of the candidate molecule and at least one binding energy of the candidate molecule on at least one conformation of the enzyme,
providing the computed molecular descriptors and each computed binding energy to a classification model trained to output, from the set of molecular descriptors and said at least one binding energy of the candidate molecule on a conformation of the enzyme, an indication about said candidate molecule being inhibitor or non-inhibitor of the enzyme, and
receiving an indication output by the classification about said candidate molecule being inhibitor or non-inhibitor of the enzyme.

In embodiments, the method for predicting whether a candidate molecule is inhibitor of a predetermined enzyme further comprises training the classification model according to the training method disclosed above.

In embodiments, the method comprises providing the computed molecular descriptors and each computed binding energy to a first classifier formed by a random forest model and a second classifier formed by a Support Vector Machine model, receiving an indication from each classifier as to whether the candidate module is inhibitor or non-inhibitor of the predetermined enzyme,
and the method further comprises:
   computing, for a plurality of conformations of the enzyme, a binding energy of the candidate molecule with each conformation of the enzyme,
   comparing the lowest computed binding energy with two thresholds and inferring, from said comparison, a third indication, and
   determining the candidate molecule as inhibitor or non-inhibitor of the enzyme according to the majority vote over the three indications.

In embodiments, the candidate molecule is a candidate drug or a xenobiotic. According to another object, a computer program product is disclosed, comprising code instructions for implementing the training or prediction methods disclosed above.

The claimed method of training a classification model comprises a step of selecting a subset of molecular descriptors, which are used as input to the classification model, wherein the molecular descriptors comprise physicochemical parameters of the considered molecule, and at least one binding energy on at least one conformation of the determined enzyme.

The selection of the descriptors is based on the relative importance of the descriptors in predicting inhibition of the enzyme; hence the number of descriptors is reduced and so is the computational time for computing the descriptors for a given molecule.

In addition, using physicochemical parameters of a molecule instead of ECFP allows a better understanding of the inhibiting factors of molecules. Last, the model can take into account various binding energies computed for different conformations of the enzyme, for increased performance. However, the binding energies are also submitted to descriptors selection and only those energies having high importance for predicting inhibition are kept.

### BRIEF DESCRIPTION OF THE DRAWINGS

Other features and advantages of the invention will be apparent from the following detailed description given by way of non-limiting example, with reference to the accompanying drawings, in which:

FIG. 1 a schematically shows the main steps of a training method according to an embodiment,

FIG. 1b schematically shows a computing device configured for implementing the training method according to an embodiment, and/or a method for predicting inhibiting character of a molecule using a trained classification model.

FIG. 2 shows the average balanced accuracy in % over 100 random forests with multiple sets of descriptors for CYP2C9, CYP2D6, SULT1A1, SULT1A3 and UGT1A1.

### DETAILED DESCRIPTION OF AT LEAST ONE EMBODIMENT

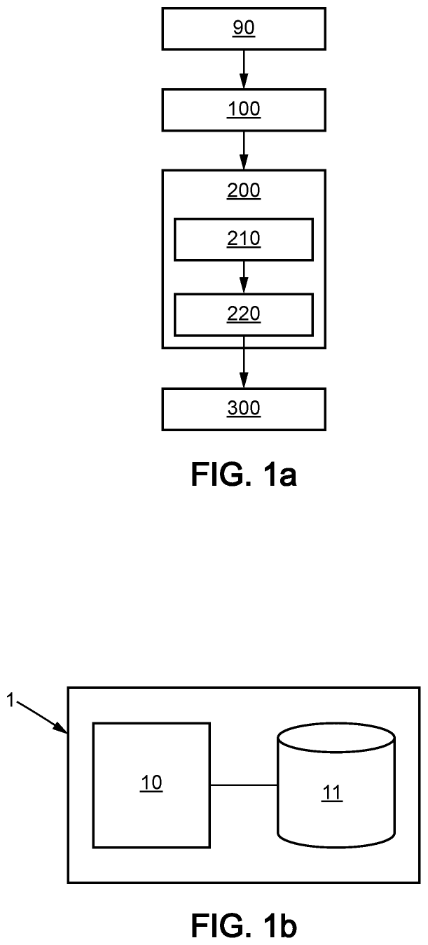

A method for training a model for predicting inhibitors of a determined drug metabolizing enzyme (DME) will now be described. With reference to FIG. 1b, the method is implemented by a training device 1 comprising a computer 10, for instance a processor, microprocessor, controller or microcontroller, and a memory 11 storing a learning dataset composed of a training and a validation datasets, and code instructions for implementing the method described above when they are executed by the computer. The training and validation datasets comprise lists of known inhibitors and non-inhibitors of the determined enzyme.

The considered DME belongs to the family of cytochromes P450 (CYPs), sulfotransferases (SULTs) or UDP-glucuronosyltransferases (UGTs). More preferably, the enzyme is one of the following group:

CYP 2C9
CYP 2D6
SULT 1A1,
SULT 1A3, and
UGT 1A1.

Thus, the method disclosed below is performed for a given enzyme among this group, and the obtained model is specific to predicting inhibition of said enzyme.

With reference to FIG. 1a, the method may comprise a preliminary step 90 of preparing learning dataset, which comprises collecting known inhibitors and non-inhibitors of the determined enzyme from literature or databases, such as ChEMBL, PubChem, BRENDA, Aureus Sciences, or TOXNET. In addition, if the number of found inhibitors or non-inhibitors is important, a selection may be performed to keep most active inhibitors. The inhibiting character of a molecule is given by an index which corresponds to the concentration of the molecules which gives a certain percentage of deactivation of an enzyme. In order to select the most active inhibitors, only those molecules causing 50% inhibition of an enzyme at a concentration inferior or equal to 100 can be selected. This is denoted AC50(IC)≤10 μM. On the other hand, a selection may be performed to keep only the least inhibiting molecules, such as the molecules showing less than 10% inhibition at 50 μM concentration (AC10(IC)<50 μM). Chemical diversity with similarity cutoff of 0.8 was employed. The centroids were then used to constitute the training and test sets.

Once this set has been obtained, an external validation dataset can be built by randomly taking 20% of both inhibiting and non-inhibiting molecules in the dataset, and the remaining 80% are kept as training dataset for the model.

As disclosed in more details below, the prediction model is a classification model which is configured to receive as input a number of descriptors computed on a given molecule, and to output a classification of the molecule as being inhibitor or non-inhibitor of the determined enzyme.

The method comprises a step 100 of building an initial set of molecular descriptors, and a selection 200 of a sub-set of molecular descriptors among this initial set, based on their relative importance in predicting the inhibiting character of a molecule.

The initial set of molecular descriptors comprises physicochemical molecular descriptors, representing features of the molecules such as its size, mass, bulkiness, volume, shape, structural symmetry and complexity, flexibility, elements, charges and bonds, bonds strength, polarity, electronegativity, polarizability, ionization potential, aromaticity, lipophilicity, surface area, polar surface area.

In embodiments, the physicochemical descriptors comprise 2D physicochemical descriptors, which are numerical properties computed from a connection table representation of a molecule and which are not dependent on the conformation of the molecule. For instance, these descriptors can be calculated using PaDEL software.

The initial set of molecular descriptors may comprise an initial number of at least 100 physicochemical descriptors, for instance at least 500 physicochemical descriptors, for instance between 500 and 2000 physicochemical descriptors.

The initial set of molecular descriptors also comprises at least one binding energy on at least one conformation of the determined enzyme. In embodiments, at least one structure of the enzyme may be selected from known databases, including for instance an apo structure and/or at least one holo co-crystallized structure, and molecular dynamics simulations may be run for each structure in order to generate different conformations of the enzyme. For instance, the CHARMM or NAMD software may be used for conformations generation.

The binding energy of a molecule on each conformation of the enzyme may be computed by performing docking of the molecule on the respective conformation. A software such as AutoDock Vina may be used for this purpose.

Preferably, the initial set of descriptors comprises a plurality of binding energies on several conformations of the enzyme. For instance, the initial set of descriptors may comprise between 1 and 20 binding energies, preferably between 2 and 15 binding energies, for instance between 2 and 10 binding energies. This allows taking into account in a same classification model different conformations of the considered enzyme. These binding energies enter in the selection of the final descriptors via calculation of the Gini importance.

The method then comprises a selection 200 of a subset of descriptors among this initial set.

The selection step 200 may comprise a preliminary step 210 of removing, from the initial set of descriptors:
descriptors having missing or infinite values on all the data of the training dataset;
descriptors with near null variance over the training dataset—a threshold may be set of removing descriptors having a variance below said threshold,
highly correlated descriptors (for instance with an absolute value of Pearson correlation coefficient superior or equal to 0.85, for instance superior or equal to 0.9)

The selection 200 of the descriptors then comprises a step 220 of selecting descriptors based on their relative importance in predicting the inhibiting character of a molecule.

In embodiments, the selection of the subset of descriptors comprises training a plurality of random forest models on the training dataset, and selecting the subset of descriptors having highest Gini importance.

Considering a binary classification problem with X₁, . . . , Xₚ as the independent variables—corresponding here to the descriptors—and Y as the response variable, where Y∈[0,1], at a given node t of a given tree T composing a random forest, the Gini index is defined as:

G(t)=2pₜ(1−pₜ) where pₜ=P(Y=0|node=t)

The Gini index, also known as Gini impurity index, is a measure of the probability of incorrectly classifying a randomly chosen element in a dataset if it were randomly labeled according to the class distribution in the dataset.

When training a decision tree or a random forest, the best split at a given node is chosen by maximizing the decrease of Gini index at the node. If variable Xⱼ splits node t to two sub-nodes t₁ and t₂, the decrease of Gini index at t is defined as:

$$Δ G ( j , t , T ) = G ( t ) - \frac{{n}_{1}}{{n}_{t}} G ( {t}_{1} ) - \frac{{n}_{2}}{{n}_{t}} G ( {t}_{2} )$$

Where nₜ is the number of sample subjects at node t, n₁ is the number of sample subjects at node t₁ and n₂ is the number of sample subjects at node t₂. The Gini importance of variable Xⱼ is:

$${I}_{G} ( j ) = {\sum }_{T} {\sum }_{t} Δ G ( j , t , T )$$

Hence the method may comprise the computation of the Giny importance of each descriptor of the initial set of descriptors (from which have been removed some of the descriptors at the end of step 210), the ranking of the descriptors according to their Gini importance, and the selection of a number of descriptors having highest Gini importance. In embodiments, a plurality of random forests, for example between several hundreds and a thousand random forests may be calculated, and the Gini importance of each descriptor may be averaged, so that the differences in randomness between models do not affect the prediction accuracy.

It should be noted that the subset of descriptors selected at the end of step 220 may no longer comprise binding energies, if the latter have lower importance than physicochemical descriptors in predicting inhibition. However, results provided below show that for each of the enzymes CYP2C9, CYP2D6, SULT1A1, SULT1A3 and UGT1A1, a plurality of binding energies does remain at the end of step 220.

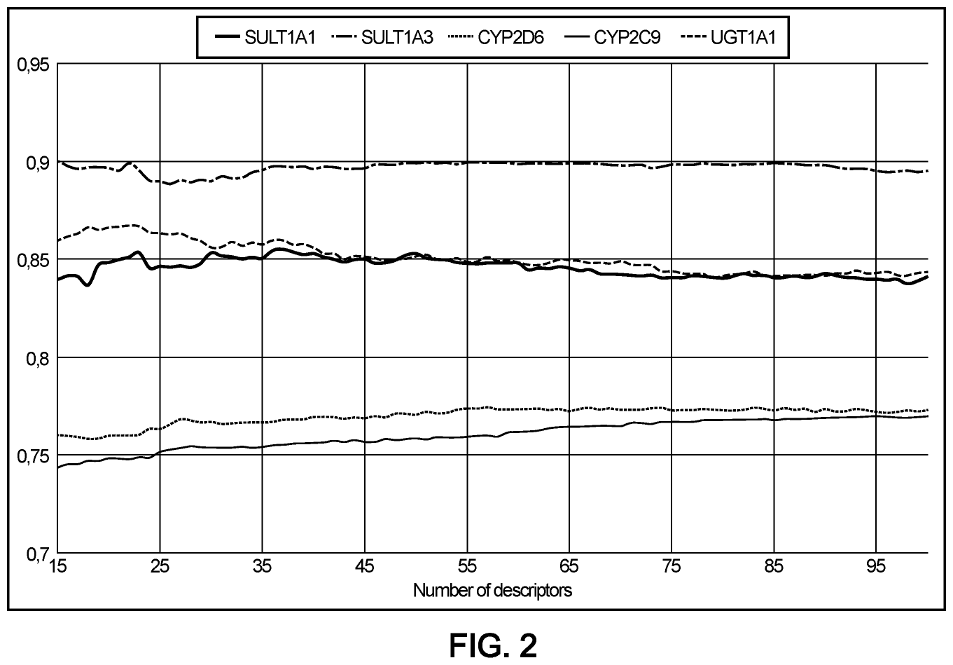

The subset of descriptors that is selected at the end of step 220 may comprise less than a hundred descriptors, for instance between 50 and 100 descriptors. In embodiments, the determination of the number of descriptors to keep at the end of step 220 may comprise calculating the performance of random forests calculated with multiple sets of descriptors from the first top 10 to the first 100 descriptors. The calculated performance may be the averaged balanced accuracy which is the mean of sensitivity and specificity. With reference to FIG. 2 is shown, on the ordinate axis, the average balanced accuracy over 100 random forest with multiple sets of descriptors having a number of descriptors, in abscissa axis, from the first 10 to the first 100 descriptors, for enzymes CYP2C9, CYP2D6, SULT 1A1, SULT1A3 and UGT1A1. The average balanced accuracy is computed on the training dataset. One can notice that the balanced accuracy for SULT 1A1 and CYP2D6 increases until between 40 and 60 descriptors and then reaches a plateau, or even slightly diminishes for CYP2D6. The number of descriptors may thus be set as the one maximizing the balanced accuracy.

In addition to improving the accuracy of the prediction model, decreasing the number of descriptors play an important role in the speed and precision of calculation of the descriptors. For instance, starting with a set of molecular descriptors comprising 382 represents a computational time of 8 minutes for computing all descriptors for one molecule. When using a selection of 83 descriptors, the computational time is reduced to 1 minute per molecule.

The same applies to the computational time for binding energies calculations, since the computational time for one molecule is multiplied by the number of different protein conformations involved and corresponding to the number of binding energies to compute for the classification model.

Once the subset of descriptors has been selected, the method comprises training 300 a classification model configured to receive as input the selected subset of descriptors, and to output a classification of a given molecule as inhibiting or non-inhibiting of the considered enzyme.

The classification model is either a Random Forest model or a Support Vector Machine Model. The model is trained by a supervised training over the learning database, i.e. for each molecule of the training dataset, the selected subset of descriptors are computed for the molecule, and an indication of the inhibiting or non-inhibiting character of the molecule on the determined enzyme is provided to the classification model.

In the embodiment where the classification model is a Random Forest model, a plurality of decision trees are built based on bootstrap samples from the training dataset, and a small subset of descriptors is randomly selected to take decisions at each node of each tree. The final classification of the random forest is obtained by taking the results of all trees by a majority vote.

The number of descriptors at each node of each tree may be equal to √p as widely accepted in the field, where p is the number of descriptors in the subset of descriptors selected at the end of step 200. Furthermore, a plurality of random forest models may be trained, with a variable number of trees within the random forest (for instance between 25 and 1024), and the classification model may be selected as the one with the number of trees providing best internal accuracy.

In the embodiment where the classification model is a Support Vector Machine, the descriptors selected at the end of step 200 can be preliminarily centered on a mean of 0 and scaled to a variance equal to 1. In embodiments, the SVM model is based on the Radial Basis Function kernel. Parameter tuning can be performed by grid search, with the cost parameter being optimized in the range of 2⁻²-2²⁰, and the gamma/sigma parameters are varied in the range of 2⁻²⁰-2².

In embodiments, step 300 comprises training both a Random Forest model and a Support Vector Machine model.

The parameters of the trained classification model may be stored in the memory.

Once the classification model has been trained, it may be used for predicting the inhibiting character of a molecule, which can be a candidate drug molecule. Testing of molecule then comprises computing the subset of selected descriptors for the given molecule and feeding the trained model with the computed descriptors, so that the trained model classifies that the molecule as inhibiting or non-inhibiting.

In embodiments where both a Random Forest model and a Support Vector Machine model have been trained, the prediction of the inhibiting character of a molecule may be performed by taking a majority vote over:
the SVM model,
the Random Forest model, and
a third classifier that is the lowest of the calculated binding energies computed for the different protein conformations of a Drug-Metabolizing enzyme, which is compared to at least one, and preferably two thresholds.

According to this last classifier, a molecule may be assigned as non-inhibitor if the corresponding binding energy (the lowest among the different protein conformations) is greater than a first threshold T1, and as inhibitor if the binding energy is lower than a second threshold T2<T1. No decision is taken if the binding energy is between T1 and T2. Using the lowest binding energy for the different enzyme conformations allows finding the enzyme conformation which is most appropriate to accommodate a ligand of interest. The generated docking position with the best ranked score (binding energy) for this ligand allows to gain information on the enzyme-ligand interaction on the atomic level.

Thus in such embodiments the prediction method may comprise, in addition to feeding the trained SVM and Random Forest models with the computed descriptors, the additional steps of:
computing, for a plurality of conformations of the enzyme, a binding energy of the candidate molecule with each conformation of the enzyme,
comparing the lowest computed binding energy with two thresholds and inferring, from said comparison, a third indication, and
determining the candidate molecule as inhibitor or non-inhibitor of the enzyme according to the majority vote over the three indications.

The computing device for computing the descriptors and applying the trained model may be the same, or may be distinct from, the training device mentioned above.

### Example

Known inhibitors and known-inhibitors of CYP2C9 were obtained from databases and only the inhibitors with AC50(IC)≤100 where kept, and non-inhibitors showing <20% inhibition at 500 concentration where kept. The training dataset resulted in 3811 inhibitors and 2468 non-inhibitors. For CYP2D6, the training dataset resulted in 343 inhibitors and 3002 non-inhibitors. For SULT1A1, 87 inhibitors and 500 decoys non-inhibitors were retained. For SULT1A3, 76 inhibitors and 370 decoys non-inhibitors were retained. For UGT1A1, 71 inhibitors and 361 decoys non-inhibitors were retained.

Two X-ray CYP2C9 structures were taken from the Protein Data Bank, co-crystallized with losartan, 5XXI, and 1R90, co-crystallized with flurbiprofen. A number of seven conformations including two crystal and five protein centroid structures with diverse binding pocket conformations were generated from previously performed MD simulations and accessible in Louet, M.; Labbe, C. M.; Fagnen, C.; Aono, C. M.; Homem-de-Mello, P.; Villoutreix, B. O.; Miteva, M. A., Insights into molecular mechanisms of drug metabolism dysfunction of human CYP2C9*30. PLoS One 2018, 13 (5), e0197249.

For CYP2D6, six conformations were generated. Two X-ray structures were taken from the Protein Data Bank, one co-crystallized with prinomastat, 3QM4, and an apo structure, 2F9Q. A number of six conformations including two crystal and four protein centroid structures with diverse binding pocket conformations were generated from previously performed MD simulations and accessible in Martiny V Y, Carbonell P, Chevillard F, Moroy G, Nicot A B, Vayer P, Villoutreix B O, Miteva M A., Integrated structure- and ligand-based in silico approach to predict inhibition of cytochrome P450 2D6. Bioinformatics. 2015, 31(24):3930-7.

For SULT1A1, 9 conformations were generated. One X-ray structure was taken from the Protein Data Bank, 4GRA. In addition, two protein centroid structures with the cofactor PAP and six protein centroid structures with the cofactor PAPS were generated from previously performed MD simulations.

For SULT1A3, 13 protein centroid structures with the cofactor PAPS were generated from previously performed MD simulations starting from the X-ray structure taken from the Protein Data Bank, 2A3R.

For UGT1A1, 10 protein centroid structures with the cofactor UDP-glucuronic acid were generated from previously performed MD simulations starting from a homology model of the substrate and the co-factor binding domains.

A number of 1050 2D physicochemical molecular descriptors were calculated on the training and validation datasets with PaDEL software. Some descriptors were removed according to step 210, in particular by removing descriptors with an absolute value of Pearson correlation coefficient higher than 0.9, resulting in a number of 382 remaining physicochemical descriptors. To these descriptors were added the binding energies for each conformation. The selection of most important descriptors according to step 220 was then performed. While FIG. 2 represents the evolution of average balanced accuracy over 100 random forest with the number of descriptors, for CYP2D6, CYP2C9, SULT1A1, SULT1A3 and UGT1A1, Table 1 below indicates the averaged balanced accuracy in % over 100 random forests including all 382 physicochemical descriptors and binding energy descriptors, i.e. prior to descriptor selection:

TABLE 1

| | Balanced Accuracy of | |
|---|---|---|
| | 382 PaDEL and binding | Number of binding |
| Protein DME | energies descriptors | energies included initially |
| CYP 2C9 | 0.775 | 7 |
| CYP 2D6 | 0.767 | 6 |
| SULT 1A1 | 0.809 | 9 |
| SULT 1A3 | 0.854 | 13 |
| UGT 1A1 | 0.810 | 10 |

Finally, it was chosen the top 88 descriptors including 5 binding energies for CYP2C9, the top 88 descriptors including 5 binding energies form CYP2D6, the top 60 descriptors including 4 binding energies for SULT1A1, the top 85 descriptors including 5 binding energies for SULT1A3, and the top 86 descriptors including 6 binding energies for UGT1A1.

Random Forest classification was performed using Random Forest R library in the statistical software package R. Random forest calculations were run scanning over a range of number of trees ntree of 25-1024 and a range in the number mtry of descriptors per node of 5-18. For each model, the combinations of the ntree and mtry parameters with best internal accuracy were selected. A second scan was done over the parameter sampsize of RandomForest in R software, which allows to choose the number of positive/negative molecules to take for each tree in case on unbalanced training dataset.

In table 2 are shown the final Random Forest models prediction accuracy in % and their corresponding parameters. The balanced accuracy is the mean of sensitivity and specificity.

TABLE 2

| RF | CYP2C9 | CYP2D6 | SULT1A1 | SULT1A3 | UGT1A1 |
|---|---|---|---|---|---|
| Internal balanced | 77.80 | 78.01 | 91.98 | 92.17 | 89.56 |
| accuracy (on | | | | | |
| training set) | | | | | |
| Internal sensitivity | 81.48 | 74.34 | 91.95 | 95.48 | 91.81 |
| Internal specificity | 74.11 | 81.68 | 92.00 | 88.87 | 87.31 |
| External balanced | 87.63 | 77.67 | 92.93 | 91.02 | 90.51 |
| accuracy (on | | | | | |
| validation test set) | | | | | |
| External sensitivity | 87.53 | 74.79 | 95.45 | 84.21 | 88.89 |
| External specificity | 87.73 | 80.56 | 90.40 | 97.83 | 92.13 |
| ntree | 256 | 300 | 256 | 256 | 256 |
| mtry | 17 | 14 | 23 | 24 | 22 |
| sampsize | 2227/2450 | 320/260 | 76/61 | 69/56 | 62/50 |
| Inhibitors/non- | | | | | |
| inhibitors | | | | | |
| Number of binding | 5 | 5 | 4 | 5 | 6 |
| energies remained | | | | | |
| in the final models | | | | | |

Support Vector Machine models were also created using the radial kernel implemented in the R package with e 1071 and Caret libraries. Parameter tuning was done by grid search using ten-fold validation repeated five times. The cost parameter was optimized in the range 2⁻²-2²⁰ and the gamma/sigma varied from 2⁻²⁰-2². To compensate highly the unbalanced dataset, a weight parameter was used which penalized misclassified observables.

In table 3 are shown the final SVM models prediction accuracy in % and their corresponding parameters:

TABLE 3

| SVM | CYP2C9 | CYP2D6 | SULT1A1 | SULT1A3 | UGT1A1 |
|---|---|---|---|---|---|
| Internal balanced | 79.10 | 78.90 | 92.56 | 93.99 | 91.27 |
| accuracy (on | | | | | |
| training set) | | | | | |
| Internal sensitivity | 82.32 | 78.78 | 91.60 | 93.00 | 86.57 |
| Internal specificity | 75.87 | 79.03 | 93.52 | 94.97 | 95.95 |
| External balanced | 84.99 | 80.51 | 94.03 | 92.01 | 95.54 |
| accuracy (on | | | | | |
| validation test set) | | | | | |
| External sensitivity | 81.17 | 79.06 | 95.45 | 89.47 | 94.44 |
| External specificity | 88.80 | 81.97 | 92.60 | 94.57 | 96.63 |
| cost | 2 | 128 | 16 | 32 | 64 |
| gamma | 0.015625 | 0.000977 | 0.00390625 | 0.001953125 | 0.00195312 |
| SVM weights | 0.4/0.6 | 0.9/0.1 | 0.9/0.1 | 0.9/0.1 | 0.8/0.2 |
| Actives/Inactives | | | | | |
| Number of binding | 5 | 5 | 4 | 5 | 6 |
| energies remained | | | | | |
| in the final models | | | | | |

One can notice that both models provide higher balanced accuracy for CYP2D6 that the approach discussed in the prior art section. In particular, the selected descriptors allow obtaining higher accuracy and increased computational speed for testing the inhibiting character of a molecule.

As indicated above, in addition to the final SVM and RF models, the lowest of the calculated binding energies for the different protein conformations of a DME, may serve as a third classifier. That allows the final decision for a molecule to be assigned as inhibitor or non-inhibitor of a DME as taking the major vote over the SVM model, RF model and the energy decision.

Using the calculated binding energies as a third classifier,
the thresholds for CYP2C9 and CYP2D6 can be −7.0 kcal/mol and −8.5 kcal/mol, hence according to this classifier it is decided that a molecule is non-inhibitor if its binding energy is >−7.0 kcal/mol and that it is inhibitor if its binding energy is <−8.5 kcal/mol:
the thresholds for SULT1A1 and SULT1A3 may be −5.0 kcal/mol and −7.5 kcal/mol, hence according to this classifier it is decided that a molecule is non-inhibitor if its binding energy is >−5.0 kcal/mol and that it is inhibitor if its binding energy is <−7.5 kcal/mol:
the thresholds for UGT1A1 may be −6.5 kcal/mol and −8.0 kcal/mol, hence according to this classificator it is decided that a molecule is non-inhibitor if its binding energy is >−6.5 kcal/mol and that it is inhibitor if its binding energy is <−8.0 kcal/mol.

1. A method for training a model for predicting inhibitors of a determined CYP, SULT or UGT enzyme, the method being implemented by a training device comprising a computer and a memory storing a training dataset comprising a plurality of molecules known as being an inhibitor or non-inhibitor of the determined CYP, SULT or UGT enzyme, the method comprising:
selecting, from an initial set of molecular descriptors comprising physicochemical molecular descriptors and at least one binding energy on at least one conformation of the determined CYP, SULT or UGT enzyme, a subset of molecular descriptors, based on the relative importance of the descriptors in predicting the inhibiting character of a molecule, and
performing a supervised training, over the training dataset, of a classification model configured to receive as input a vector formed of the subset of molecular descriptors computed on a molecule, and to output an indication of the inhibiting character of the molecule on the determined CYP, SULT or UGT enzyme.

2. The method according to claim 1, wherein the determined CYP, SULT or UGT enzyme is selected from the group consisting of:
CYP 2C9
CYP 2D6
SULT 1A1,
SULT 1A3, and
UGT 1A1.

3. The method according to claim 1, wherein selecting the subset of descriptors based on their relative importance comprises training a plurality of random forest models on the training dataset, computing a Gini importance index of all descriptors of the set, and selecting the molecular descriptors having highest Gini importance.

4. The method according to claim 3, wherein determining the number of descriptors to select based on their relative importance comprises computing an average balanced accuracy of a plurality of random forest models with multiple sets of descriptors having a varying number of descriptors, and selecting the number of descriptors maximizing the average balanced accuracy.

5. The method according to claim 3, comprising, prior to the step of selecting, a step of removing, from the initial set of molecular descriptors:
highly correlated descriptors;
descriptors having missing or infinite values on data of the training dataset; and
descriptors having a variance below a determined threshold over the training dataset.

6. The method according to claim 1, wherein the classification model is a random forest model or a Support Vector Machine model.

7. A classification model configured for predicting whether a molecule is an inhibitor of a predetermined enzyme, wherein the classification model is obtained by training a training dataset in accordance with the method of claim 1.

8. The classification model according to claim 7, wherein the classification model comprising:
a first classifier formed by a random forest model trained according to 7;
a second classifier formed a Support Vector Machine model trained according to 7; and
a third classifier indicating whether a molecule is an inhibitor of the predetermined enzyme based on the comparison of the lowest binding energy computed for a plurality of conformations of the predetermined enzyme with at least one threshold;
the output of the model being the major vote over the three classifiers.

9. A method for predicting whether a candidate molecule is an inhibitor of a enzyme, comprising:
computing a set of molecular descriptors of the candidate molecule and at least one binding energy of the candidate molecule on at least one conformation of the enzyme,
providing the computed set of molecular descriptors and the at least one computed binding energy to a classification model trained to output, from the set of molecular descriptors and said at least one binding energy of the candidate molecule on a conformation of the enzyme, an indication output about whether said candidate molecule is an inhibitor or non-inhibitor of the enzyme, and
receiving the indication output by the classification model about whether said candidate molecule is an inhibitor or non-inhibitor of the enzyme.

10. The method of claim 9, further comprising training the classification model by
selecting, from an initial set of molecular descriptors comprising physicochemical molecular descriptors and at least one binding energy on at least one conformation of the enzyme, a subset of molecular descriptors based on the relative importance of the molecular descriptors in predicting the inhibiting character of a molecule, and
performing a supervised training using a training dataset of a classification model configured to receive as input a vector formed of the subset of molecular descriptors, and to output an indication of whether the candidate molecule is an inhibitor or non-inhibitor of the enzyme.

11. The method according to claim 9, wherein the step of providing comprises
providing the set of molecular descriptors and each computed binding energy to a first classifier formed by a random forest model and a second classifier formed by a Support Vector Machine model,
receiving an indication from each classifier as to whether the candidate module is an inhibitor or non-inhibitor of the predetermined enzyme,
computing, for a plurality of conformations of the enzyme, a binding energy of the candidate molecule with each conformation of the enzyme,
comparing the lowest computed binding energy with two thresholds and inferring, from said comparison, a third indication, and
determining whether the candidate molecule is an inhibitor or non-inhibitor of the enzyme according to the majority vote over the three indications.

12. The method of claim 9, wherein the candidate molecule is a candidate drug or a xenobiotic.

13. (canceled)

14. A non-transitory computer-readable support having stored thereon code instructions which, when executed by a computer, cause the computer to carry out the method according to claim 1.
